(19) United States
(12) Patent Application Publication
(10) Pub. No.: US 2017/0128401 A1
(43) Pub. Date: May 11, 2017

(54) DIETARY COMPOSITION FOR IMPROVED PERFORMANCE

(71) Applicants: Jason Daniel Dooney, Tinton Falls, NJ (US); Daniel Alexander Mosner, Jersey City, NJ (US)

(72) Inventors: Jason Daniel Dooney, Tinton Falls, NJ (US); Daniel Alexander Mosner, Jersey City, NJ (US)

(21) Appl. No.: 15/343,620

(22) Filed: Nov. 4, 2016

Related U.S. Application Data
(60) Provisional application No. 62/251,639, filed on Nov. 5, 2015.

(51) Int. Cl. A61K 31/20; A61K 36/258; A61K 31/685; A61K 36/53; A23L 33/12; A61K 31/202; A61K 31/353; A61K 9/00; A61K 31/522; A23L 2/52; A61K 36/16; A61K 36/69

(52) U.S. Cl. CPC A61K 31/20; A61K 36/16; A61K 36/258; A61K 31/685; A61K 36/53; A61K 36/69; A61K 31/202; A61K 31/353; A61K 9/0095; A61K 9/0056; A61K 31/522; A23L 2/52; A23L 33/12; A23V 2002/00

(57) ABSTRACT

A beverage or food additive for improving an individual's mental and physiological performance, the dietary composition comprising medium chain triglycerides oil, Ginko Biloba, Panax Ginseng, phosphatidylserine, Bacopa Monnieri, and Polygala Tenuifolia. In addition, the dietary composition may optionally contain docosahexaenoic acid (DHA), eicosapentaenoic acid (EPA), and/or epigallocatechin gallate (EGCG).

This application claims the benefit of U.S. Provisional Application No. 62/251,639 filed Nov. 5, 2015.

### FIELD OF THE INVENTION

The present invention relates to a dietary composition directed to increasing mental and physiological performance in individuals when the composition is consumed directly or as a beverage or food additive.

### BACKGROUND

Coffee and tea are some of the most widely consumed beverages around the world and typically a staple of an individual's morning routine. Individuals typically consume coffee or tea to, among other purposes, stimulate or enhance brain and/or physical performance in preparation for any number of activities, such as interacting with other people, improve overall physical and/or mental energy levels, and for general enhancement of ability to function in carrying out daily tasks, work, and so forth.

Medium Chain Triglycerides (MCT) oil is a widely used additive in coffee or tea for providing an individual with an influx or rush of physiological energy coupled with an elevation or boost of mental alertness or performance. MCT oil is directly absorbed and rapidly oxidized for energy and may increase the use of fats as energy, resulting in a high level of lipolysis and an increase in metabolism. MCTs have been used to potentially alter an athlete's reliance on carbohydrates for fuel and enhance an athlete's reliance on fat for energy, thereby providing an important performance enhancer, particularly for athletes who consume low levels of carbohydrates. Use of MCTs with caffeine found in coffee or tea may result in providing quickly and easily absorbed energy from the MCTs coupled with increased attention and focus from the caffeine. However, the mental performance enhancement effects of caffeine typically dissipate as an individual's tolerance of caffeine increases with repeated use. Accordingly, individual users of MCT oil as a coffee or tea additive often must drink several cups of coffee to retain a baseline level of alertness.

Despite the potential performance enhancing capability of MCTs as an additive to coffee or tea, there remains a need in the art for an improved dietary composition capable of enhancing an individual's mental and physiological performance. Moreover, there remains a need for such a dietary composition that can be consumed directly or as an additive to either a beverage or a food. The present dietary composition provides such a desirable composition.

### SUMMARY OF THE INVENTION

The presently disclosed dietary composition is directed to a nutritional composition that includes, but is not limited to, MCT oil, Ginko Biloba, Panax Ginseng, Phosphatidylserine, Bacopa Monnieri, and Polygala Tenuifolia. In addition, the additive may optionally contain docosahexaenoic acid (DHA) and eicosapentaenoic acid (EPA), as well as epigallocatechin gallate (EGCG). The composition may be consumed alone or as an additive to a beverage or food.

The present dietary composition is formulated for a single serving or administration to contain 8-30 g of MCT oil, 120-360 mg of Ginko Biloba, 200-400 mg of Panax Ginseng, 200-750 mg of phosphatidylserine, 100-300 mg of Bacopa Monnieri, and 100-300 mg of Polygala Tenuifolia. The dietary composition may be consumed alone, or typically will be used as a beverage additive to coffee or tea. Alternatively, or in combination, the dietary composition may be used as a food additive, particularly as an additive to butter.

The dietary composition disclosed herein will be administered to an individual in a method of improving mental and physiological performance in the individual. Ingestion of the present formulation(s) may result in increased performance in a number of respects, including, but not limited to, attention, alertness, and cognition; reduction of anxiety and fatigue; decreased forgetfulness; increased ability to grasp concepts; increase of spatial processing and organizational tasks; increased mental processing speed and accuracy; improved mood and social functioning; and an increase in anaerobic endurance.

Some of the objectives of the invention having been stated, other objectives will appear as the description proceeds.

### DETAILED DESCRIPTION OF THE PREFERRED EMBODIMENTS

While the present technology is capable of various embodiments, there is described in detail specific embodiments with the understanding that the present disclosure exemplifies the principles of the technology and is not intended to limit the technology to the embodiments disclosed.

As used herein, the term “dietary composition” refers to a composition that is meant to be consumed or ingested by an individual, either alone/directly or as an additive to a beverage or food. The dietary composition may also be referred to as a “dietary supplement” or “supplement” that is intended to be consumed as a supplement to an individual's overall diet. The term “additive” refers to a composition that is intended to be added to a beverage or food for consumption. The term “nutritional” refers to a dietary composition or additive that has nutritional value in that it can provide or assist in providing a source of physiological energy. In addition, the dietary composition disclosed herein improves or increases mental performance when used alone or as an additive.

The presently disclosed dietary composition may be consumed directly or added to any number of beverages and foods. In a preferred embodiment, the dietary composition may be an additive that is added to a caffeinated beverage, such as coffee or tea. These caffeinated beverages are typically consumed in the morning, at or near the start of an individual's day, and help provide increased mental awareness or performance, as well as a physiological boost in energy. The present dietary composition may be added to coffee, tea or other caffeinated beverage to provide a significantly improved increase in mental performance compared to that achieved by drinking the beverage alone. In this regard, the present dietary composition can serve as a creamer in coffee or tea that upon consumption or ingestion delivers a boost in mental performance coupled with a boost in energy.

In another embodiment, the present dietary composition can be an additive that is added to food to deliver increased mental awareness or performance, as well as physiological energy. In particular, the dietary composition may be added to butter, which is often consumed as a spread in the morning. The dietary composition also combines well with butter, given its fat content. Overall, when consumed as an additive to a beverage or food consumed at or near the start of an individual's day, the dietary composition typically will be used to provide a boost in physiological energy and mental performance.

The presently disclosed nutritional dietary composition improves upon MCT oil as a performance enhancing dietary composition that provides physiological energy coupled with an elevation or boost of mental alertness or performance. In particular, five separate cognitive and/or performance enhancing herbal components are added to MCT oil: Ginko Biloba, Panax Ginseng, phosphatidylserine, Bacopa Monnieri, and Polygala Tenuifolia. The components provide synergistic benefits, with the advantages of each of the components detailed herein. The components may likewise leverage or enhance effects of caffeine or other inherent, beneficial components in beverages or food, such as the polyphenol epigallocatechin-3-gallate (EGCG) found in green tea. Ingestion of the five components dissolved in MCT oil may increase or improve mental and/or physiological performance, including, but not limited to, attention, alertness, and cognition; reduce anxiety and fatigue, decrease task forgetfulness, increase ability to grasp concepts, increase spatial processing and organizational tasks, increase mental processing speed and accuracy, improve mood and social function, and may increase anaerobic endurance. In addition to these components, the disclosed dietary composition may include the optional components docosahexaenoic acid (DHA) and eicosapentaenoic acid (EPA), which have been associated with improved cognition and brain function. Furthermore, the dietary composition may include the optional component epigallocatechin gallate (EGCG), which has been associated with decreased carbohydrate absorption and increased endurance.

Some of the components in the disclosed dietary composition utilize MCT oil as a solvent. For example, Bacopa Monnieri and phosphatidylserine require a fat based solvent. The water solubility of MCT oil enables it to serve as a carrier of these components into a water based beverage such as coffee or tea. Likewise, MCT oil can serve as a carrier of these components into butter or similar food as an additive.

The presently disclosed dietary composition is consumed directly or added to coffee, tea or butter, typically in increments or single serving amounts having 8-30 g of MCT oil. The following amounts of the above components are dissolved into the 8-30 g increment of MCT oil: 120-360 mg of Ginko Biloba, 200-400 mg of Panax Ginseng, 200-750 mg of phosphatidylserine, 100-300 mg of Bacopa Monnieri, and 100-300 mg of Polygala Tenuifolia. In addition, the dietary composition may include the optional components DHA, EPA, and/or EGCG. Typically, the amounts of these components dissolved into an 8-30 g increment of MCT oil will be 40 mg DHA, 80 mg EPA, and/or 260-950 mg of EGCG.

MCT oil is a composition of medium chain triglycerides of 6-12 carbons in length, and is found in coconut oil, palm kernel oil, and dairy fat (such as that in cheese, butter, milk and yogurt). The relatively low carbon length of MCTs coupled with their water solubility change the metabolism and absorption of the MCT oil compared to oils having long chain triglycerides with more than 12 carbons in length. The liver is able to oxidize the MCT oils for quick energy. MCT oil does not require the amino acid transporter L-carnitine to transport the fats across the mitochondrial membrane, unlike other fat sources. MCT oil has been shown to provide quick energy, improve mobilization of fat stores, increase metabolism, and spare lean body mass (Benardot, D., 2012, Advanced Sports Nutrition (2^{nd} ed.)).

The nutritional formula of the dietary composition presently disclosed comprises 8-30 g of MCT oil per serving. The MCT oil utilized in the dietary composition is typically derived from either coconut oil, palm oil, dairy fat, or another source containing substantial amounts of MCT oil. The MCT oil is a composition of medium chain triglycerides of 6-12 carbons in length: six carbons (C6), caproic acid; C8, caprylic acid; C10, capric acid; and C12, lauric acid. The MCT oil used in the present dietary composition typically will contain, but is not limited to, naturally occurring proportions of these four MCT acids. Alternatively, MCT oil used in the present dietary composition may be processed to contain predominantly one or more forms of medium chain triglycerides. In this regard, a MCT oil containing a single MCT, for example caprylic acid; two MCTs, for example caprylic acid and capric acid; and three MCTs, for example caprylic acid, capric acid; and lauric acid may be used in the dietary composition. Accordingly, the presently disclosed dietary composition may incorporate any single MCT or combinations thereof in the MCT oil utilized for a specific formulation.

In the presently disclosed dietary composition, MCT oil acts as a main solvent for the fat-soluble components Bacopa Monnieri and phosphatidylserine. Bacopa Monnieri and phosphatidylserine require a lipid transporter to be absorbed and thus, the MCT oil enhances the absorption of these components and their oral effectiveness.

Phosphatidylserine as used in the nutritional formulas of the present dietary composition can be derived from any of a number of sources, and is typically derived from a soy lecithin, bovine, or another substantial source of phosphatidylserine. Phosphatidylserine as used in the presently disclosed formulas is at a concentration of 200-750 mg of phosphatidylserine per 8-30 g of MCT oil.

Phosphatidylserine has a structure similar to triglyceride with two fatty acid chains bonded to a glycerol molecule and the third glycerol binding site bonded to a phosphatic acid molecule. Given the fat solubility of phosphatidylserine, it has been posited that supplementation with a fatty acid source such as medium chain triglycerides can improve results of enhanced brain function and anaerobic endurance obtained upon phosphatidylserine ingestion. The present dietary composition utilizes the fat-soluble properties of MCT oil to improve phosphatidylserine uptake and its effectiveness.

Phosphatidylserine has been clinically shown to enhance brain function and increase anaerobic endurance. Enhanced brain function was observed in clinical studies in which administration of 400 mg of phosphatidylserine to subjects was shown increase speed of calculations by 20% and improve test accuracy, with a 13% increase in correct answers and a 39% decrease in wrong answers. In studies of anaerobic endurance, administration of a 750 mg dose of phosphatidylserine has been shown to increase time to exhaustion in anaerobic events (85% VO2 max) by 29%+/−8% (Phosphatidylserine, Scientific Review on Usage, Dosage, Side effects; https://examine.com/supplements/phosphatidylserine/; retrieved Oct. 14, 2016).

The nutritional formula of the presently disclosed dietary composition comprises a Ginko Biloba obtained from a 50:1 concentrated extract source to provide a standardized terpenoid and flavonoid composition. The Ginko Biloba is standardized to contain 22-27% flavone glycosides, 5-7% terpene lactones; which includes a 2.8-3.4% content of ginkgolides A-C, 2.6-3.2% bilobalide, and the ginkgolic acids are kept below 5 ppm (5 μg/g dry weight) (Ginko Biloba, Scientific Review on Usage, Dosage, Side effects; https://examine.com/supplements/ginkgo-biloba/; retrieved Oct. 14, 2016). The concentration of Ginko Biloba in the formula of the present dietary composition formula is 120 mg-360 mg per 8-30 g of MCT oil.

Ginko Biloba has been shown to have both an anti-stress effect and to contribute to improved cognitive performance. 120 mg of a Ginko Biloba supplementation that was administered before a stress test was found to have an anti-stress effect and attenuate spikes in blood pressure and cortisol. Supplementation with Ginko Biloba attenuates the effects of corticosterone, which affects the processes of memory that are induced by stressful situations.

In a double blind study on 20 young adult subjects who were 21.2 years median age old, 360 mg of Ginko Biloba, 400 mg of Panax Ginseng, and a 960 mg combination were tested on measures of cognition. The quality of memory as determined by accuracy of all memory tasks was improved with each treatment, but most significantly in the 960 mg combination. Ginko Biloba alone induced impairment in working recall but this effect was not present with the addition of Panax Ginseng. Secondary memory as defined by accuracy of immediate and delayed word call, picture, and word recognition tasks was also enhanced for all three treatments. The 960 mg combination of Ginko Biloba and Panax Ginseng improved accuracy of spatial and numeric working memory tasks (Kennedy, D. et.al, 2002, Physiology & Behavior 75: 739-751). Speed of attention is enhanced at 240-360 mg of Ginko Biloba. The present dietary composition utilizes the combination of Ginko Biloba and Panax Ginseng to improve memory while its use as a coffee additive aids in speed of attention.

A low dose of Ginko Biloba extract (120 mg) failed to show significant correlation with cognitive performance benefit in some studies. However, supplementation alongside phosphatidylserine enhances the bioactive components of Ginko Biloba extract. The combination of Ginko Biloba extract and phosphatidylserine increased the effectiveness of their cognitive enhancing effects relative to a placebo or Ginko Biloba alone (Kennedy, D. et.al, 2007, Hum. Psychopharmacol. 22(4):199-210). Participants in the aforementioned study showed improvements in secondary memory performance, speed of memory tasks, and increased calmness. The present dietary composition utilizes phosphatidylserine to potentiate the cognitive enhancing effect of Ginko Biloba at a low dose in the MCT oil.

The nutritional formula of the currently disclosed dietary composition comprises Panax Ginseng extract that contains 4-10% ginseonsides. The concentration of Panax Ginseng in the present invention is 200-400 mg per 8-30 mg MCT oil.

The present dietary composition contains both Panax Ginseng and Polygala Tenuifolia. Consumption of Panax Ginseng with Polygala Tenuifolia has been suggested to increase circulating levels and absorption of Panax Ginseng.

Panax Ginseng has been shown to improve mood and mental performance. In a double blind study of 18-29 year old subjects, 30 participants were given doses of 0, 200, and 400 mg of Panax Ginseng. The group administered 400 mg demonstrated increases in calmness and increased mental arithmetic after 8 days of Panax Ginseng supplementation (Reay, J. et.al, 2010, Hum Psychopharmacol. 25(6):462-71).

Consumption of 200 mg of Panax Ginseng has been shown to increase cognition, social function, and mood after 4 weeks of supplementation relative to a placebo (Ellis, J. and Reddy, P., 2002, Ann Pharmacother. 36(3):375-9). The present dietary composition's inclusion of Panax Ginseng gives rise to improvement in morning cognition and overall mood after consumption of the dietary composition's composition of MCT oil and the components added to it.

Administration of 400 mg of Panax Ginseng has been shown to improve mental efficiency in a serial sevens subtractions task (Kennedy, D. et.al, 2002, Physiology & Behavior 75: 739-751). As mentioned above, the combination of Panax Ginseng and Ginko Biloba in the present dietary composition formula have been shown to improve spatial reaction time, improve accuracy, and reduce quantity of errors on cognitive tests.

The nutritional formula of the presently disclosed dietary composition comprises 100-300 mg of Bacopa Monnieri or the equivalent of 6 g of dry herb with a total bacoside content of 55% by weight.

Consumption of Bacopa Monnieri may increase memory formation by regulating the enzyme tryptophan hydroxylase to increase the expression of the serotonin transporter. Supplementation with Bacopa Monnieri for 4 weeks has been shown to initiate neural growth to increase dendritic intersections and branching points in areas that coincide with memory. These growths may make Bacopa Monnieri effective in improving short term memory encoding and decreasing short term forgetfulness, increasing retention speeds, improving memory, and improving verbal learning and delayed recall (Bacopa Monnieri, Scientific Review on Usage, Dosage, Side effects, https://examine.com/supplements/bacopa-monnieri/, retrieved Oct. 14, 2016).

Bacopa Monnieri may regulate dopamine spikes in the striatum as induced with caffeine. Regulation of dopamine spikes has been indicated to decrease caffeine based locomotion (i.e., a jittery feeling). The anti-dopaminergic effects exhibited by Bacopa Monnieri may also be useful in addressing drug dependence, including addiction to caffeine. This potential regulation of dopamine does not affect the anti-sleep effects of caffeine and can assist individuals in consuming a smaller amount of caffeine throughout the day by reducing drug dependence.

In a double blind study of healthy individuals of all ages, 300 mg of Bacopa Monnieri supplementation over 12 weeks was shown to improve speed of early information processing, verbal learning rate and memory consolidation (Stough, C., et.al, 2001, Psychopharmacology (Berl)., 156(4):481-4). Administration of Bacopa Monnieri positively affects the integrity of early information processing which can act as a rate limiting process for cognition. Supplementation of Bacopa Monnieri also decreases task based forgetting and through improved information processing, increases the ability to grasp concepts in the long term.

The nutritional formula of the present invention comprises 100-300 mg of Polygala Tenuifolia per 8-30 g of MCT oil. The Polygala Tenuifolia may be added to the MCT oil in an ethanolic extract.

Administration of Polygala Tenuifolia may increase compounds that improve brain growth such as Nerve Growth Factor and Brain-Derived Neutrophic Factor. Ingestion of active substances contained within Polygala Tenuifolia can increase hippocampal activity (Polygala Tenuifolia, Scientific Review on Usage, Dosage, Side effects, https://examine.com/supplements/polygala-tenuifolia/, retrieved Oct. 14, 2016).

In a clinical study of healthy middle aged adults, administration of 300 mg of Polygala Tenuifolia improved recall and recognition. Participants also noted significant improvements in spatial memory and organization, with a 19% reduction in errors relative to placebo (Lee, J., et. al, 2009, Neurosci Lett., 454(2):111-4). In view of these effects, Polygala Tenuifolia is a potent component in the presently disclosed dietary composition MCT oil formulations by significantly contributing to improvement of spatial awareness upon ingestion of the dietary composition. Accordingly, Polygala Tenuifolia contributes to the present dietary composition's utility in improving the efficiency of individual's morning routines.

The presently disclosed dietary composition may optionally include the long-chain omega-3 fatty acids DHA and EPA. These omega-3 fatty acids have been implicated in providing cognitive benefits, as studies with DHA and EPA have shown that consumption of these omega-3 fatty acids can contribute to improved attention, reaction time, cognitive performance, and mood in young, healthy adults (Kidd, P., 2007, Alternative Med. Rev., 12:207-227). Accordingly, addition of DHA and EPA to the present dietary composition may work in conjunction with the other five components to impart a greater or more robust improvement in mental performance.

The presently disclosed dietary composition may further optionally include the green tea catechin epigallocatechin gallate (EGCG), either alone or in combination with DHA and EPA. This Green Tea Catechin has been associated with increased VO₂ₘₐₓ and significantly decreased carbohydrate absorption in healthy adults (Richards et. al, 2010, Med. Sci. Sports Exerc. 42:739-744; Lochocka et. al, 2015, Sci. Rep., 5:12015). Accordingly, addition of EGCG to the present additive may work in conjunction with the other five components to impart a greater improvement in physical endurance and improve the maintenance of a low carb, ketogenic lifestyle.

In view of the above, the presently disclosed dietary composition provides a composition of MCT oil with the components Ginko Biloba, Panax Ginseng, phosphatidylserine, Bacopa Monnieri, and Polygala Tenuifolia, with the optional ingredients DHA and EPA. This dietary composition provides a composition that taken alone or as a beverage or food additive enhances or improves mental performance while supplying physiological energy. Accordingly, a method of improving mental and physiological performance in an individual comprises administering an effective amount of the presently disclosed dietary composition. The improvement in mental and physiological performance includes, but is not limited to, increased cognition, awareness, recall, memory, anaerobic performance, social function, improvement in calculative tasks, the ability to grasp concepts, and decreased stress. These parameters of mental and physiological performance are typically inhibited or slow for individuals in the morning or at the start of their day. The present dietary composition will therefore be particularly useful for consumption alone or as a beverage or food additive for individuals in the morning or at the start of their day. In this respect, the dietary composition will typically be used in a caffeinated beverage, such as coffee or tea. Alternatively, or in combination, the dietary composition may be used as a food additive. In this aspect, the dietary composition will typically be used as an additive in butter or a similar food.

In a preferred embodiment, a method of improving mental and physiological performance in an individual comprises administering an effective amount of dietary composition containing MCT oil, Ginko Biloba, Panax Ginseng, phosphatidylserine, Bacopa Monnieri, and Polygala Tenuifolia. In yet another embodiment, the dietary composition further contains docosahexaenoic acid and eicosapentaenoic acid. In one embodiment, the effective amount of dietary composition per serving or administration is formulated to contain 8-30 g of MCT oil, 120-360 mg of Ginko Biloba, 200-400 mg of Panax Ginseng, 200-750 mg of phosphatidylserine, 100-300 mg of Bacopa Monnieri, and 100-300 mg of Polygala Tenuifolia. In addition to the amounts of these components, a preferred formulation of the dietary composition may include 40 mg of docosahexaenoic acid, 80 mg eicosapentaenoic acid, and 260-950 mg of epigallocatechin gallate (EGCG).

As indicated above, the present dietary composition will be useful when consumed alone or as a beverage or food additive for individuals in the morning or at the start of their day. Accordingly, in a preferred method of improving mental and physiological performance in an individual, an effective amount of the present dietary composition will be administered to an individual as an additive in a beverage. Preferably, the beverage is a caffeinated beverage. Typically, the presently disclosed method will entail adding an effective amount of the present dietary composition (as detailed above) to a cup or per serving of coffee or tea, which is then consumed by an individual. In this regard, the presently disclosed method will significantly boost or improve the gain in mental performance individuals typically experience upon consumption of coffee or tea. Thus, in the presently disclosed method, the dietary composition can effectively serve as a creamer in coffee or tea that upon consumption or ingestion delivers a boost in mental performance coupled with a boost in energy.

In addition to use of the dietary composition in a beverage, the presently disclosed methods further include use of the dietary composition in a food to administer an effective amount of the preferred formulation per serving. In a preferred embodiment, a method of improving mental and physiological performance in an individual entails administering an effective amount by adding the dietary composition to butter, which is then consumed by the individual. An individual can therefore derive the benefits of the present dietary composition by consuming it in butter, which is often consumed as a spread for foods commonly eaten in the morning.

While the presently disclosed beverage and food dietary composition has been described as having particular components and amounts disclosed herein, the present technology can be further modified within the spirit and scope of this disclosure. This application is therefore intended to cover any variations, uses, or adaptations of the technology using its general principles. Further, this application is intended to cover such departures from the present disclosure as come within known or customary practice in the art to which this technology pertains and which fall within the limits of the appended claims.

### EXAMPLE I

The effectiveness of the disclosed dietary composition at improving mental and physiological performance in an individual is examined in a two-week double blind, placebo controlled study crossover design using resistance trained athletes. Subjects are administered daily a dietary composition containing 8-30 g of MCT oil, 120-360 mg of Ginko Biloba, 200-400 mg of Panax Ginseng, 200-750 mg of phosphatidylserine, 100-300 mg of Bacopa Monnieri, and 100-300 mg of Polygala Tenuifolia, or a placebo. A specific amount of each of the components is chosen and consistently administered throughout the study. At zero, one and two weeks, a profile of mood states, serial subtraction tests, breath by breath respiratory data, and blood samples are obtained from the subjects. The data from these studies is used to assess mood, working memory, speed of calculations, fat oxidation, and exercise to exhaustion. Subjects administered the dietary composition are predicted to have statistically significant improvement in mood, working memory, speed of calculations, fat oxidation, and exercise to exhaustion parameters over those subjects administered placebo.

### EXAMPLE II

The effectiveness of the disclosed dietary composition at improving mental and physiological performance in an individual is examined in a twelve-week double blind, placebo controlled study crossover design using resistance trained athletes. Subjects are administered daily a dietary composition containing 8-30 g of MCT oil, 120-360 mg of Ginko Biloba, 200-400mg of Panax Ginseng, 200-750 mg of phosphatidylserine, 100-300 mg of Bacopa Monnieri, and 100-300 mg of Polygala Tenuifolia, or a placebo. A specific amount of each of the components is chosen and consistently administered throughout the study. At zero, six and twelve weeks, subjects are examined with psychological tests and self-ordered pointing tests, and profile of mood states are obtained from the subjects. The data from this trial is used to assess memory, cognition, mood, and subjective well-being. Subjects administered the dietary composition are predicted to have statistically significant improvement in memory, cognition, mood, and overall well-being.

What is claimed is:

1. A dietary composition comprising medium chain triglycerides (MCT) oil, Ginko Biloba, Panax Ginseng, phosphatidylserine, Bacopa Monnieri, and Polygala Tenuifolia.

2. The dietary composition of claim 1 formulated to comprise per serving 8-30 g of MCT oil, 120-360 mg of Ginko Biloba, 200-400 mg of Panax Ginseng, 200-750 mg of phosphatidylserine, 100-300 mg of Bacopa Monnieri, and 100-300 mg of Polygala Tenuifolia.

3. The dietary composition of claim 1, further comprising docosahexaenoic acid and eicosapentaenoic acid.

4. The dietary composition of claim 2, further formulated to comprise 40 g of docosahexaenoic acid and 80 g of eicosapentaenoic acid.

5. The dietary composition of claim 1, further comprising epigallocatechin gallate.

6. The dietary composition of claim 2, further formulated to comprise 260-950 mg of epigallocatechin gallate.

7. The dietary composition of claim 1, wherein the dietary composition is a beverage additive.

8. The dietary composition of claim 7, wherein the beverage is a caffeinated beverage.

9. The dietary composition of claim 1, wherein the dietary composition is a food additive.

10. The dietary composition of claim 9, wherein the food is butter.

11. A method of improving mental and physiological performance in an individual comprising administering an effective amount of a dietary composition comprising medium chain triglycerides (MCT) oil, Ginko Biloba, Panax Ginseng, phosphatidylserine, Bacopa Monnieri, and Polygala Tenuifolia.

12. The method of improving mental and physiological performance of claim 11, the effective amount of the dietary composition comprising 8-30 g of MCT oil, 120-360 mg of Ginko Biloba, 200-400 mg of Panax Ginseng, 200-750 mg of phosphatidylserine, 100-300 mg of Bacopa Monnieri, and 100-300 mg of Polygala Tenuifolia.

13. The method of improving mental and physiological performance of claim 11, wherein the dietary composition further contains docosahexaenoic acid and eicosapentaenoic acid.

14. The method of improving mental and physiological performance of claim 12, wherein the effective amount of the dietary composition further comprises 40 g of docosahexaenoic acid and 80 g of eicosapentaenoic acid.

15. The method of improving mental and physiological performance of claim 11, wherein the dietary composition further comprises epigallocatechin gallate.

16. The method of improving mental and physiological performance of claim 12, wherein the effective amount of the dietary composition further contains 260-950 mg epigallocatechin gallate.

17. The method of improving mental and physiological performance of claim 11, wherein the dietary composition is administered as an additive to a beverage.

18. The method of improving mental and physiological performance of claim 17, wherein the beverage is a caffeinated beverage.

19. The method of improving mental and physiological performance of claim 11, wherein the dietary composition is administered as an additive to a food.

20. The method of improving mental and physiological performance of claim 19, wherein the food is butter.
